(19) United States
(12) Patent Application Publication
(10) Pub. No.: US 2020/0338228 A1
(43) Pub. Date: Oct. 29, 2020

(54) LIGHT-RESPONSIVE PRESSURE SENSITIVE ADHESIVES FOR WOUND DRESSINGS

(71) Applicant: KCI LICENSING, INC., San Antonio, TX (US)

(72) Inventors: Prathamesh Madhav KHARKAR, San Antonio, TX (US); Timothy Mark ROBINSON, Blandford Forum (GB); Diwi L. ALLEN, San Antonio, TX (US); Marisa Annette SCHMIDT, San Antonio, TX (US)

(73) Assignee: KCI LICENSING, INC., San Antonio, TX (US)

(21) Appl. No.: 16/762,450

(22) PCT Filed: Nov. 13, 2018
(86) PCT No.: PCT/US2018/060718

§ 371 Date: May 7, 2020

Related U.S. Application Data
(60) Provisional application No. 62/585,315, filed on Nov. 13, 2017.

(51) Int. Cl. A61L 15/58; C09J 9/00; C09J 7/38; A61L 15/26

(52) U.S. Cl. CPC A61L 15/58; C09J 9/00; C09J 7/38; A61L 15/26; C09J 2301/302; A61F 13/0266; C09J 2301/502; C09J 2475/00; C09J 2433/00; C09J 2483/00; C09J 2203/37

(57) ABSTRACT

Disclosed are photo-responsive pressure sensitive adhesives, wound dressings that contain the photo-responsive pressure sensitive adhesives, and methods to remove the wound dressing. The photo-responsive pressure sensitive adhesive comprises a viscoelastic polymeric matrix. The viscoelastic polymeric matrix comprises a polymer that includes at least a first polymeric segment and at least a second polymeric segment that are each covalently linked together through a photo-cleavable moiety. The photo-cleavable moiety is capable of photolytic cleavage of the covalent bonds formed between the moiety and the first and/or second polymeric segments, respectively, in response to electromagnetic radiation.

### CROSS-REFERENCE TO RELATED APPLICATIONS

This application claims priority to and the benefit of U.S. Provisional Application No. 62/585,315, filed Nov. 13, 2017, the entire contents of which is incorporated herein by reference.

### FIELD OF INVENTION

The present invention concerns photo-responsive pressure sensitive adhesives that can be used on wound dressings and methods of removing the wound dressings from tissue sites of wounds. The tack of the photo-responsive pressure sensitive adhesives can be reduced in response to electromagnetic radiation, thereby alleviating the pain and discomfort caused by removing wound dressings from tissue sites of wounds.

### BACKGROUND OF THE INVENTION

Pressure sensitive adhesives, due to ease of application, are commonly used for attaching wound dressings or wound drapes to tissue sites of wounds. Over the last two decades, great progress has been made in developing pressure sensitive adhesives that can securely bond wound dressings to tissue sites through day-to-day activities. However, pain and discomfort caused by removing the wound dressings remain the main drawbacks of these pressure sensitive adhesives. Furthermore, due to the adhesive strength of theses pressure sensitive adhesives, the removal of wound dressings can damage the regenerated or unhealed tissue on the wound, resulting in regression of the wound healing process. To alleviate these problems, recently a few adhesives have been developed, in which the adhesive strength can be reduced in response to stimuli to facilitate removal of wound dressings from wounds.

For instance, Hurwitz et al. (U.S. Pat. No. 7,396,976) discloses an easy-to-peel bandage comprising an adhesive layer that contains adhesive-inactivating mineral oil or vegetable oil enclosed in microcapsules. Upon applying pressure or being scratched, the microcapsules rupture and the mineral oil or vegetable oil is released, thereby inactivating the adhesive. However, the pressure and/or scratching required for rupturing the microcapsules can cause severe pain to the sensitive skin and tissues on the tissue site of a wound, defeating the purpose of inactivating the adhesives.

Tunius et al. (PCT application No. PCT/GB2014/000236) discloses a light-responsive adhesive. The adhesive strength of the light responsive-adhesive can be reduced via free radical polymerization of acrylates in the presence of suitable photoinitiators when the adhesive is exposed to light. While Tunius et al.'s adhesive facilitates removal of the wound dressings from a wound, it nevertheless suffers several drawbacks. First, the photoinitiators, which are used to initiate the polymerization of acrylates, can trigger uncontrolled and undesirable photo-polymerization with biological molecules at the tissue sites, thereby lowering the effectiveness of the adhesive strength reduction by polymerization of acrylates and interfering with biological activity at the wound sites. Second, the photoinitiators can also cause irritation on the wound sites due to their low biocompatibility. Moreover, the free radical polymerization can be triggered by stray light and/or ambient light, resulting in premature reduction of adhesive strength before the wound dressing needs to be removed.

Robinson et al. (U.S. Pat. No. 8,450,554 B2) introduces a system for healing a wound at tissue site. Robinson et al.'s system comprises an adhesive with micelles dispersed therein and an adhesive-weakening material encapsulated in the micelles. The adhesive-weakening material can be released into the adhesive in response to light, thus reducing the bond strength between the wound dressing and the tissue site of the wound. However, Robinson et al.'s system is relatively complex to manufacture, as it requires the adhesive-weakening material to be encapsulated in light-decomposable micelles in a liquid tight manner. Moreover, these micelles have to be evenly dispersed throughout the adhesive layer to ensure uniform tack reduction on the tissue site. Therefore, the costs for manufacturing such wound healing systems can be relatively high.

Overall, while adhesives and adhesive-agent systems exist for facilitating painless removal of wound dressings, the need for improvements in this field persists in light of at least the aforementioned drawbacks for these adhesives and adhesive-agent systems.

### SUMMARY OF THE INVENTION

A solution to at least some of the above-mentioned problems associated with removal of wound dressings has been discovered. The solution resides in a photo-responsive pressure sensitive adhesive comprised of a polymer that undergoes polymer scission in response to electromagnetic radiation of a certain/targeted wavelength. The polymer scission reduces tack of the photo-responsive pressure sensitive adhesive. This can be beneficial for reducing pain associated with removing the wound dressing from a wound site. Notably, the polymer scission can be initiated in response to a targeted wavelength so as to reduce or avoid instances where incident sunlight or stray light source (e.g. ambient indoor light) initiates polymer scission. By way of example, the adhesive of the present invention can be designed to initiate polymer scission in response to light having a wavelength in the UV spectrum or higher energy visible light spectrum or both. This can allow for the use of an artificial light source to initiate polymer scission rather than incident sunlight. Still further, this process can be initiated in a medical facility setting and/or directly by the patient outside of such a setting (e.g., at home, place of work, etc.) and can be done with reduced agitation of the wound. Therefore, the adhesives of the present invention provide a technical achievement over at least some of the problems associated with the currently available adhesives mentioned above, such as low biocompatibility, irritation of wound tissue, high production costs, and/or inadvertent simulation.

Certain embodiments of the invention are directed to photo-responsive pressure sensitive adhesives. In certain aspects, the photo-responsive pressure sensitive adhesive may comprise a viscoelastic polymeric matrix of a polymer that includes at least a first polymeric segment and at least a second polymeric segment that are each covalently linked together through a photo-cleavable moiety. The photo-cleavable moiety is capable of photolytic cleavage of the covalent bonds formed between the moiety and the first and/or second polymer segments, respectively in response to electromagnetic radiation. This cleavage can result in polymer scission and ultimately a reduction in the adhesive strength of the adhesive. The electromagnetic radiation may have a desired or targeted wavelength. In certain instances, the wavelength of the radiation can be 300 nm to 500 nm, preferably 300 nm to 400 nm, or more preferably about 365 nm. However, it is contemplated in the context of the present invention that other wavelength ranges or specific wavelengths can be used to initiate cleavage of the photo-cleavable moiety, including lower energy visible light, infrared light, microwaves, and/or radio waves. The photolytic cleavage of the covalent bonds formed between the moiety and the first and second polymeric segments may occur within 1 second to 500 seconds, preferably within 5 seconds to 360 seconds, in response to the targeted/selected electromagnetic radiation. In certain aspects of the present invention, the viscoelastic polymeric matrix can be cytocompatible and/or biocompatible to help reduce adverse reactions with the wound site.

The photo-cleavable moiety can be a substituted aromatic nitro compound. The aromatic nitro compound may have the following structure:

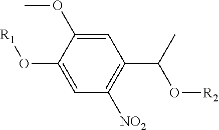

wherein R₁ and R₂ are each individually an acryloyl group, a methacryloyl group, an isocyanate group, or a hydroxyalkyl group that forms a covalent bond with the first polymeric segment and the second polymeric segment, respectively. The polymer may have a general structure of:

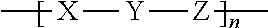

wherein X is the first polymer segment, Y is the photo-cleavable moiety, Z is the second polymeric segment, and n is 1 to 10,000.

In certain aspects, the photolytic cleavage breaks the R₁—O bond and/or the R₂—O bond. A molecular weight of each of the first polymeric segment and the second polymeric segment may be in a range of 10,000 g/mol to 1,000,000 g/mol. In some aspects, the first and second polymeric segments each can be a viscoelastic polymer. Alternatively, the first polymeric segment is a viscoelastic polymer and the second polymeric segment may be a non-viscoelastic polymer. Still further X and Z can have the same polymer structure but have different lengths/molecular weights. Alternatively, X and Z can have different polymer structures (e.g., different monomeric units). The polymer segments X and Z can each be homopolymers or copolymers. In some preferred instances, X and Z are homopolymers comprised of the same monomeric unit.

In some embodiments, non-limiting examples of the viscoelastic polymer may include a polyacrylate, a polymethacrylate, a polyurethane, a hydrogel, a natural rubber, a synthetic rubber, a styrene block copolymer, a polyvinyl ether, a polyolefin, a hydrocolloid, or a silicon or silicone based material, a styrene butadiene, a polyisoprene, a polychloroprene, a polybutadiene, a polychlorosulphonated polyethylene, a rosin tackyfier, a coumarone resin or a coumarone indene resin, and combinations thereof. In certain aspects of the invention, the viscoelastic polymeric matrix may have a glass transition temperature (Tg) of −80° C. to 70° C., preferably −80° C. to 30° C.

Certain embodiments of the invention are directed to wound dressings. In certain aspects, the wound dressings may comprise a first surface and an opposing second surface. A pressure sensitive adhesive layer comprising the photo-responsive pressure sensitive adhesive may be attached to at least a portion of the second surface of the wound dressing. In certain aspects of the invention, the wound dressing further include a releasable film attached to at least a portion of the pressure sensitive adhesive layer. The pressure sensitive adhesive layer may contain 10 g/m² to 200 g/m² of the photo-responsive pressure sensitive adhesive. The desired amount of the photo-responsive pressure sensitive adhesive in the pressure sensitive adhesive layer may be determined by the required tack level for the wound dressing.

In other more particular aspects of the invention, the wound dressing may further include a light filtering layer comprising a pigment and/or a dye attached to at least a portion of the first surface. The light filtering layer may be adapted to reflect and/or absorb electromagnetic radiation having a wavelength of 300 nm to 800 nm, preferably 400 nm to 800 nm, or more preferably 500 nm to 800 nm. Thus, the light filtering layer can be configured to prevent stray light and/or ambient light (sunlight or artificial light, such as incandescent, LED, fluorescent, etc.) from prematurely triggering the photolytic cleavage of the one or more of the covalent bonds formed between the first polymeric segment and the photo-cleavable moiety and/or between the second polymeric segment and the photo-cleavable moiety. In certain aspects, the light filtering layer is transparent. In other aspects, the light filtering layer is opaque. In embodiments of the invention, the first surface of the wound dressing is a drape layer. The wound dressing may further include an absorbent layer that is positioned between the second surface and the pressure sensitive adhesive layer.

Certain embodiments of the invention are directed to methods of removing the wound dressing. The methods can include subjecting the wound dressing to selected electromagnetic radiation such that photolytic cleavage of one or more of the covalent bonds formed between the first polymeric segment and the photo-cleavable moiety and/or between the second polymeric segment and the photo-cleavable moiety occurs. In certain aspects, the electromagnetic radiation may be ultraviolet radiation. The wavelength of the electromagnetic radiation may be in a range of 300 nm to 400 nm, preferably about 365 nm. However, and as noted above and throughout this specification, the adhesive can be designed to be responsive to other wavelength ranges. In other more particular aspects, the tack of the pressure sensitive adhesive layer can be reduced by at least 10% after being exposed to the electromagnetic radiation for 5 seconds to 360 seconds. The electromagnetic radiation may have an irradiance of 1 to 50 W/cm². The electromagnetic radiation can be in continuous or pulsed mode.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The term “biocompatible”, when used in the claims and/or the specification, means that a compound and/or a composition elicits an appropriate immunological and/or inflammatory response when applied in vivo.

The term “cytocompatible”, when used in the claims and/or the specification means that a compound and/or a composition is not toxic to mammalian cells. Cytocompatible materials should not negatively affect cell functions such as metabolism, cell membrane integrity, adhesion, protein transcription, proliferation, differentiation, and viability.

The term “viscoelastic” as that term is used in the specification and/or claims means that a material exhibits both viscous and elastic characteristics when undergoing deformation. Viscous characteristics refers to a material that can resist shear flow and strains linearly with time when a stress is applied. Elastic characteristics refers to a material that is capable of recovering from deformation to its original state when a stress applied thereon is removed.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the transitional phase “consisting essentially of,” in one non-limiting aspect, a basic and novel characteristic of the pressure sensitive adhesives of the present invention are their ability to initiate polymer scission in response to a selected electromagnetic radiation.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### DESCRIPTION OF THE DRAWINGS

For a more complete understanding, reference is now made to the following descriptions taken in conjunction with the accompanying drawings, in which:

FIGS. 1A and 1B illustrate polymer scission of polymers comprised in a photo-responsive pressure sensitive adhesive in response to electromagnetic radiation, according to embodiments of the invention; and

FIGS. 2A and 2B are schematics of a wound dressing before and after it is applied to a tissue site, according to embodiments of the invention.

### DETAILED DESCRIPTION OF THE INVENTION

The currently available adhesives or adhesive-agent systems used for painless removal of wound dressings suffer several deficiencies including low biocompatibility, irritation to wound sites, complex structures, uncontrolled tack reduction, and/or high manufacturing costs. The present invention provides a solution to at least some of these problems. The solution is premised on a photo-responsive pressure sensitive adhesive that can be used on wound dressings. The photo-responsive pressure sensitive adhesive is configured to undergo polymer scission in response to electromagnetic radiation at a certain wavelength, thereby reducing the tack of the adhesive and facilitating removal of wound dressings in a controlled manner. The photo-responsive pressure sensitive adhesive can be biocompatible and/or cytocompatible. Furthermore, the photo-responsive pressure sensitive adhesive does not require pressure or scratching applied to the wound dressing, thereby avoiding irritation of the wound site. Moreover, the wound dressings using the photo-responsive pressure sensitive adhesive have a structure that does not demand high manufacturing costs.

These and other non-limiting aspects of the present invention are discussed in further detail in the following sections.

### A. Photo-Responsive Pressure Sensitive Adhesive

Pressure sensitive adhesives are a type of adhesives that form a bond with a substrate (e.g. a tissue site of a wound) when pressure is used to apply the adhesives to the substrates without the presence of a solvent (e.g. water or other organic/inorganic solvent) or heat activation. The polymer chains of the pressure sensitive adhesives entangle across the interface between the adhesives and the substrates to form adhesion there between.

For conventional pressure sensitive adhesives, during the de-bonding process, a pulling force away from the substrate is applied to the adhesive to overcome the polymer entanglement across the interface between the adhesive and the substrate, thereby detaching the adhesive from the substrate. When a wound dressing attached to a wound via pressure sensitive adhesive is being removed, the pulling force can cause great pain and discomfort. The present invention provides a pressure sensitive adhesive for a wound dressing that requires reduced pulling force or substantially no pulling force to overcome the entanglement of the polymer chains comprised in the adhesive, thereby facilitating removal of the wound dressing.

The photo-responsive pressure sensitive adhesive of the present invention is configured to undergo polymer scission in response to electromagnetic radiation. The polymer scission can reduce the entanglement of polymer chains across the interface between the adhesive and the tissue site of a wound, thereby reducing the tack of the adhesive. The photo-responsive pressure sensitive adhesive comprises a viscoelastic matrix. A polymer comprised in the polymeric matrix includes at least a first polymeric segment and at least a second polymeric segment that are each covalently linked together through a photo-cleavable moiety. When the photo-responsive pressure sensitive adhesive is exposed to electromagnetic radiation, the photo-cleavable moiety maybe capable of photolytic cleavage of the covalent bonds formed between the moiety and the first and/or second polymer segments, respectively. Therefore, the polymer in the adhesive can be broken into polymeric segments, reducing the tack of the adhesive. In certain aspects, the electromagnetic radiation may have a wavelength in a range of 300 nm to 500 nm , preferably 300 nm to 400 nm, or more preferably about 365 nm and all ranges and values there between, including 300 nm to 310 nm, 310 nm to 320 nm, 320 nm to 330 nm, 330 nm to 340 nm, 340 nm to 350 nm, 350 nm to 360 nm, 360 nm to 370 nm, 370 nm to 380 nm, 380 nm to 390 nm, 390 nm to 400 nm, 400 nm to 410 nm, 410 nm to 420 nm, 420 nm to 430 nm, 430 nm to 440 nm, 440 nm to 450 nm, 450 nm to 460 nm, 460 nm to 470 nm, 470 nm to 480 nm, 480 nm to 490 nm, 490 nm to 500 nm. However, it is contemplated in the context of the present invention that other wavelength ranges or specific wavelengths can be used to initiate cleavage of the photo-cleavable moiety, including lower energy visible light (e.g., greater than 500 nm to 800 nm), infrared light (e.g., greater than 800 nm to 1,000 nm, preferably in the range of 960 nm to 990 nm), microwaves (e.g., 1 mm to 1 m), and/or radio waves (1 mm to 1000,000 km).

In some embodiments of the invention, the photo-cleavable moiety can be a substituted aromatic nitro compound. More specifically, the substituted aromatic nitro compound may have the following structure:

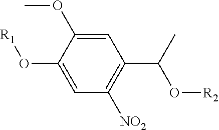

wherein R₁ and R₂ are each individually an acryloyl group, a methacryloyl group, an isocyanate group, or a hydroxyalkyl group that forms a covalent bond with the first polymeric segment and the second polymeric segment, respectively. In a photo-cleavable moiety, R₁ and R₂ may be the same or different from each other. Non-limiting examples for the photo-cleavable moiety include substituted aromatic nitro compounds (a) to (d) having the following structures:

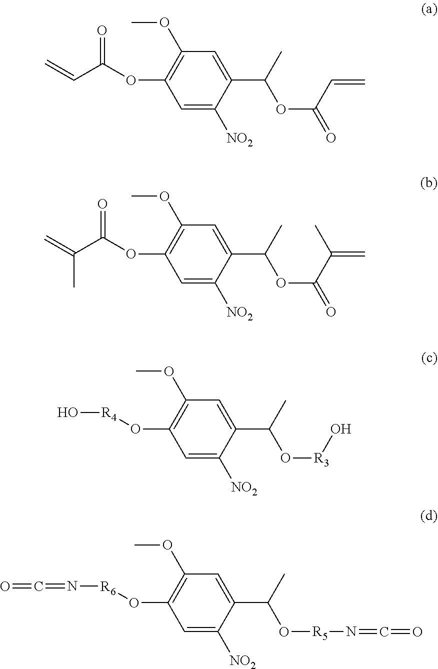

wherein R₃ and R_{4,} are each individually an alkyl group covalently bound with the adjacent two oxygen atomes in structure (c), and R₅ and R₆ are each individually an alkyl group covalently bound with the adjacent oxygen atom and nitrogen atom in structure (d). The polymer comprised in the viscoelastic polymeric matrix may have the following general structure:

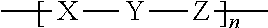

wherein X is the first polymeric segment, Y is the photo-cleavable moiety, Z is the second polymeric segment, and n is 1 to 10,000. A polymer chain of the viscoelastic polymeric matrix may comprise one or more photo-cleavable moieties. In a polymer chain with n≥1, the photo-cleavable moieties may vary from one to another repeating unit of the general structure.

In certain aspects of the invention, the first polymeric segment (X) and the second polymeric segment (Z) are each a viscoelastic polymer. Alternatively, in other aspects of the invention, the first polymeric segment (X) is a viscoelastic polymer and the second polymeric segment (Z) may be a non-viscoelastic polymer. In some embodiments of the invention, the non-limiting examples of the viscoelastic polymer of the first polymeric segment and/or the second polymeric segment may include a polyacrylate, a polymethacrylate, a polyurethane, a hydrogel, a natural rubber, a synthetic rubber, a styrene block copolymer, a polyvinyl ether, a polyolefin, a hydrocolloid, or a silicon or a silicone based material. The molecular weight of each of the first polymeric segment and the second polymeric segment may be in a range of 10,000 g/mol to 1,000,000 g/mol and all ranges and values there between, including 10,000 g/mol to 50,000 g/mol, 50,000 g/mol to 100,000 g/mol, 100,000 g/mol to 150,000 g/mol, 150,000 g/mol to 200,000 g/mol, 200,000 g/mol to 250,000 g/mol, 250,000 g/mol to 300,000 g/mol, 300,000 g/mol to 350,000 g/mol, 350,000 g/mol to 400,000 g/mol, 400,000 g/mol to 450,000 g/mol, 450,000 g/mol to 500,000 g/mol, 500,000 g/mol to 550,000 g/mol, 550,000 g/mol to 600,000 g/mol, 600,000 g/mol to 650,000 g/mol, 650,000 g/mol to 700,000 g/mol, 700,000 g/mol to 750,000 g/mol, 750,000 g/mol to 800,000 g/mol, 800,000 g/mol to 850,000 g/mol, 850,000 g/mol to 900,000 g/mol, 900,000 g/mol to 950,000 g/mol, and 950,000 g/mol to 1,000,000 g/mol. Still further, X and Z can have the same polymer structure but have different lengths/molecular weights. Alternatively, X and Z can have different polymer structures (e.g., different monomeric units). The polymer segments X and Z can each be homopolymers or copolymers. In some preferred instances, X and Z are homopolymers comprised of the same monomeric unit.

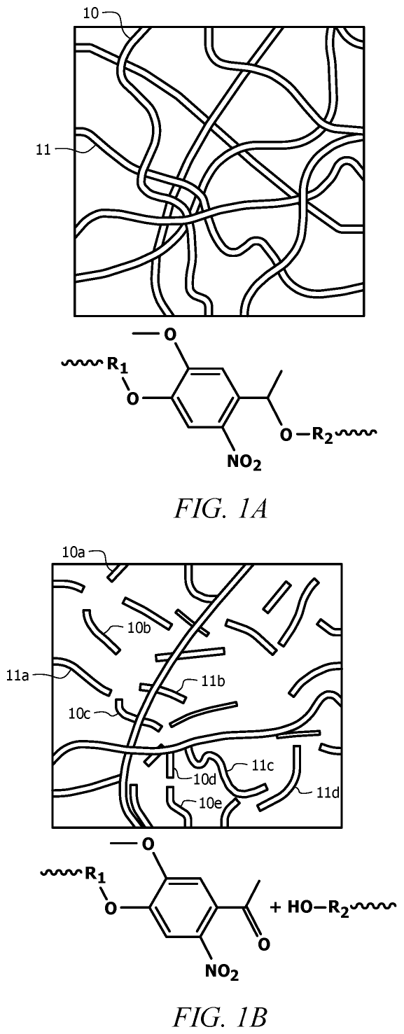

In embodiments of the invention, the photolytic cleavage may include breaking the R₁—O bonds and/or the R₂—O bonds of the photo-cleavable moieties in the polymers of the viscoelastic polymeric matrix. In some more particular aspects, the photo-cleavable moiety is capable of photolytic cleavage of the covalent bonds (R₁—O bond and/or the R₂—O bond) formed between the moiety and the first and/or second polymeric segments, respectively within 1 second to 1200 seconds, preferably 5 seconds to 360 seconds, in response to electromagnetic radiation. The breaking of the R₁—O bonds and/or the R₂—O bonds in the photo-cleavable moieties may result in polymer scission and entanglement reduction of the polymer chains of the photo-responsive pressure sensitive adhesive. FIGS. 1A and 1B demonstrate the reduction of polymer chain entanglement of the photo-responsive pressure sensitive adhesive in response to light, according to embodiments of the invention. As shown in FIG. 1A, the photo-responsive pressure sensitive adhesive includes polymer chains with high level of entanglement before they are exposed to light. For instance, polymer chain 10 and polymer chain 11 in FIG. 1A are both long polymer chains and highly entangled with other long polymer chains. After applying light to the photo-responsive pressure sensitive adhesive, at least some covalent bonds (the R₁—O bond and/or the R₂—O bond described above) formed between the moieties (Y) and the polymeric segments (X and Z) undergo photolytic cleavage, breaking at least some of the polymer chains (e.g. polymer chain 10 and polymer chain 11 of FIG. 1A) of the adhesive into polymeric segments (e.g. segments 10a to 10e, and segments 11a to 11d), as shown in FIG. 1B. The entanglement of the polymer chains in the photo-responsive pressure sensitive adhesive is reduced since the resulted polymeric segments from photolytic cleavage are significantly shorter than the polymer chains before the light exposure. Consequently, the tack of the photo-responsive pressure sensitive adhesive can be reduced.

In embodiments of the invention, the photo-responsive pressure sensitive adhesive comprising the viscoelastic polymeric matrix is cytocompatible and/or biocompatible. Therefore, the photo-responsive pressure sensitive adhesive, when used for securing a wound dressing to a wound site, can be designed to be not cytotoxic to mammalian cells and/or to not trigger significant immunological and/or inflammatory response at the wound site. In certain aspects of the invention, the viscoelastic polymeric matrix has a glass transition temperature (Tg) in a range of −80° C. to 70° C., preferably −80° C. to 30° C. or 30° C. to 70° C., and all ranges and values there between including −80° C. to −75° C., −75° C. to −70° C., −70° C. to −65° C., −65° C. to −60° C., −60° C. to −55° C., −55° C. to −50° C., −50° C. to −45° C., −45° C. to −40° C., −40° C. to −35° C., −35° C. to −30° C., −30° C. to −25° C., −25° C. to −20° C., −20° C. to −15° C., −15° C. to −10° C., −10° C. to −5° C., −5° C. to 0° C., 0° C. to 5° C., 5° C. to 10° C., 10° C. to 15° C., 15° C. to 20° C., 20° C. to 25° C., 25° C. to 30° C., 30° C. to 35° C., 35° C. to 40° C., 40° C. to 45° C., 45° C. to 50° C., 50° C. to 55° C., 55° C. to 60° C., 60° C. to 65° C., and 65° C. to 70° C. In some embodiments of the invention, the first and/or second polymeric segments in the viscoelastic polymeric matrix may include a hot melt adhesive. The glass transition temperature (Tg) of the hot melt adhesive may be 30° C. to 70° C. above a use temperature thereof. Exemplary hot melt adhesives may include, but are not limited to, polyolefins, polyamides, polyesters, Ethylene vinyl acetate based copolymers, polyurethanes, polycarbonates, silicone rubbers, polycaprolactone and combinations thereof. The use temperature of the hot melt adhesive may be in a range of 80 to 200° C.

### B. Wound Dressing Containing a Photo-Responsive Pressure Sensitive Adhesive

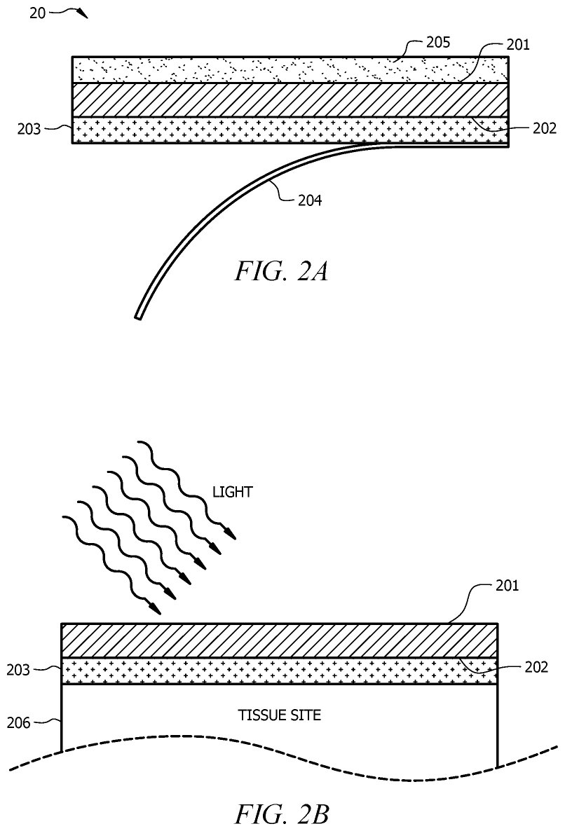

In embodiments of the invention, there are provided wound dressings that can have no or reduced pain associated with their removal from tissue sites of wounds. As illustrated in FIG. 2A, in some embodiments, wound dressing 20 may include first surface 201 and opposing second surface 202. Pressure sensitive adhesive layer 203 comprising the photo-responsive pressure sensitive adhesive, according to embodiments of the invention, may be attached to at least a portion of second surface 202 of wound dressing 20. In certain aspects, first surface 201 may be a drape layer. The drape layer may comprise a polymer. Non-limiting examples of the polymer comprised in the drape layer include polyurethane, polyamide, polypropylene, polyethylene, polyvinyl chloride, ethylene vinyl acetate copolymers, polyvinyl alcohol, polyether block amide (PEBAX) polymers, and combinations thereof. In certain aspects, wound dressing 20 may be used for a negative-pressure wound therapy. In some embodiments, wound dressing 20 may be used in a vacuum-assisted closure procedure.

In embodiments of the invention, pressure sensitive adhesive layer 203 may comprise 10 g/m² to 200 g/m² of the photo-responsive pressure sensitive adhesive, and all ranges and values there between including 10 g/m² to 20 g/m², 20 g/m² to 30 g/m², 30 g/m² to 40 g/m², 40 g/m² to 50 g/m², 50 g/m² to 60 g/m², 60 g/m² to 70 g/m², 70 g/m² to 80 g/m², 80 g/m² to 90 g/m², 90 g/m² to 100 g/m², 100 g/m² to 110 g/m², 110 g/m² to 120 g/m², 120 g/m² to 130 g/m², 130 g/m² to 140 g/m², 140 g/m² to 150 g/m², 150 g/m² to 160 g/m², 160 g/m² to 170 g/m², 170 g/m² to 180 g/m², 180 g/m² to 190 g/m², and 190 g/m² to 200 g/m². In some embodiments, pressure sensitive adhesive layer 203 comprised in wound dressing 20 may further include a non-photoresponsive pressure sensitive adhesive combined with the photo-responsive pressure sensitive adhesive. The non-photoresponsive pressure sensitive adhesive comprised in pressure sensitive adhesive layer 203 may be adapted to modulate the level of tack reduction in response to electromagnetic radiation for the pressure sensitive adhesive layer 203. Non-limiting examples of non-photoresponsive pressure sensitive adhesives include a polyacrylate, a hydrogel, a natural rubber, a synthetic rubber, a styrene block copolymer, a polyvinyl ether, a polyolefin, a hydrocolloid, a silicon or silicone based material, and combinations thereof.

In some embodiments, wound dressing 20 further includes releasable film 204 attached to at least a portion of pressure sensitive adhesive layer 203. Releasable film 204 is adapted to protect pressure sensitive adhesive layer 203 before wound dressing 20 is applied to a tissue site of a wound. In certain aspects, wound dressing 20 may further comprise light filtering layer 205. Light filtering layer 205 may include a pigment and/or a dye attached to at least a portion of first surface 201. In embodiments of the invention, light filtering layer 205 is configured to reflect and/or absorb electromagnetic radiation having a wavelength of 300 nm to 800 nm, preferably 400 nm to 800 nm, or more preferably 500 nm to 800 nm, and all ranges and values there between including 300 nm to 320 nm, 320 nm to 340 nm, 340 nm to 360 nm, 360 nm to 380 nm, 380 nm to 400 nm, 400 nm to 420 nm, 420 nm to 440 nm, 440 nm to 460 nm, 460 nm to 480 nm, 480 nm to 500 nm, 500 nm to 520 nm, 520 nm to 540 nm, 540 nm to 560 nm, 560 nm to 580 nm, 580 nm to 600 nm, 600 nm to 620 nm, 620 nm to 640 nm, 640 nm to 660 nm, 660 nm to 680 nm, 680 nm to 700 nm, 700 nm to 720 nm, 720 nm to 740 nm, 740 nm to 760 nm, 760 nm to 780 nm, and 780 nm to 800 nm. Therefore, light filtering layer 205 may be capable of preventing ambient light and/or stray light from prematurely reducing the tack of light-responsive pressure sensitive adhesive via polymer scission (photolytic cleavage of the covalent bonds formed between the photo-cleavable moiety and the first and/or second polymeric segments of the photo-responsive pressure sensitive adhesive). In some embodiments, light filtering layer 205 is transparent. Alternatively, light filtering layer 205 can be opaque. In certain aspects, light filtering layer 205 may be releaseably attached to first surface 201. Light filtering layer 205 may be released from first surface 201 before the photo-responsive pressure sensitive adhesive comprised in pressure sensitive adhesive layer 203 is exposed to electromagnetic radiation. In other aspects, light filtering layer 205 may be dissolvable in a solvent including, but not limited to, ethanol, isopropanol, acetone, or combinations thereof. Thus, light filtering layer 205 may be removed by the solvent before the photo-responsive pressure sensitive adhesive comprised in pressure sensitive adhesive layer 203 is exposed to electromagnetic radiation. As shown in FIG. 2B, pressure sensitive adhesive layer 203 may be in direct contact with tissue site 206. Light filtering layer 205 may be removed and/or released from first surface 201 of wound dressing 20 before light is applied to pressure sensitive adhesive layer 203. In some embodiments, wound dressing 20 may further include an absorbent layer (not shown) that is positioned between second surface 202 and pressure sensitive adhesive layer 203. The absorbent layer may be adapted to absorb excessive fluid on a tissue site of a wound. The excessive fluid on a tissue site may include blood, wound exudate, or a combination thereof.

### C. Method of Removing Wound Dressing

In embodiments of the invention, there are provided methods of removing aforementioned wound dressings, which contain the photo-responsive pressure sensitive adhesives. In some embodiments, a method of removing wound dressing 20 may include subjecting wound dressing 20 to electromagnetic radiation such that photolytic cleavage of one or more of the covalent bonds formed between the first polymeric segment and the photo-cleavable moiety and/or between the second polymeric segment and the photo-cleavable moiety occurs. In certain aspects, the electromagnetic radiation may be ultraviolet radiation. The electromagnetic radiation may have a wavelength in a range of 300 nm to 400 nm, or preferably about 365 nm, and all ranges and values there between, including 305 nm, 310, nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, and 395 nm. However, and as noted above, the pressure sensitive adhesives of the present invention can be designed to undergo polymer scission in response to other wavelength ranges and specific wavelengths. This “tuning” of the wavelength can be obtained by selecting an appropriately responsive photo-cleavable moiety to be used in the adhesives of the present invention.

In embodiments of the invention, the tack of pressure sensitive adhesive layer is reduced by at least 10% after being exposed to electromagnetic radiation for a duration of 1 seconds to 1200 seconds, preferably 5 seconds to 360 seconds, and all ranges and values there between, including 5 seconds to 35 seconds, 35 seconds to 65 seconds, 65 seconds to 95 seconds, 95 seconds to 125 seconds, 125 seconds to 155 seconds, 155 seconds to 185 seconds, 185 seconds to 215 seconds, 215 seconds to 245 seconds, 245 seconds to 275 seconds, 275 seconds to 305 seconds, 305 seconds to 335 seconds, and 335 seconds to 360 seconds. Preferably, the duration of the electromagnetic radiation exposure may be in a range of 10 seconds to 60 seconds. In certain aspects, the electromagnetic radiation may have an irradiance of 1 to 50 W/cm² and all ranges and values there between. In some embodiments, the method of removing wound dressing 20 may further include removing light filtering layer 205 before subjecting wound dressing 20 to the electromagnetic radiation. In certain aspects, the removing of light filtering layer 205 may include dissolving light filtering layer 205 using a solvent. As described above, non-limiting solvent may include ethanol, isopropanol, acetone, or combinations thereof. In other aspects, the removing of light filtering layer may comprise peeling releasable light filtering layer 205 from first surface 201 of wound dressing 20.

### D. Method of Making Pressure Sensitive Adhesives and Wound Dressings Having the Same

In embodiments of the invention, there are further provided methods of producing aforementioned photo-responsive pressure sensitive adhesives and wound dressings. The method of producing the photo-responsive pressure sensitive adhesives may include synthesizing the polymers comprised in the above-mentioned viscoelastic polymeric matrix. In certain aspects, the polymers comprised in the above-mentioned viscoelastic polymeric matrix can be synthesized by covalently linking at least a first polymeric segment and at least a second polymeric segment together through the photo-cleavable moiety.

In some more particular embodiments, the photo-cleavable moiety may be the substituted aromatic nitro compound with the above-mentioned structure. At least one of the first and the second polymeric segments can be a viscoelastic polymer. The viscoelastic polymer can include a polyacrylate, a polymethacrylate, a polyurethane, or a silicone rubber. In certain aspects, a synthesis scheme for the polymers of the photo-responsive pressure sensitive adhesives can include free radical polymerization (including reversible-deactivation radical polymerization), ionic polymerization, coordination polymerization, polycondensation, polyaddition polymerization, or polymerization via urethane chemistry.

In embodiments of the invention, the method of producing the photo-responsive pressure sensitive adhesive may further include mixing and compounding the photo-responsive pressure sensitive adhesives using the polymers that are synthesized via the synthesis scheme described above. In certain aspects, the mixing and compounding may include mixing the synthesized polymers of the photo-responsive pressure sensitive adheisve with a non-photoresponsive pressure sensitive adhesive comprising polyacrylate, polymethacrylate, polyurethane, silicone rubber, or combinations thereof. The weight ratio of the polymers of the photo-responsive pressure sensitive adhesive to the non-photoresponsive pressure sensitive adhesive may be determined by a desired level of tack reduction in response to electromagnetic radiation. In certain aspects, when such mixture of photo-responsive and non-responsive pressure sensitive adhesive is used, the amount of photo-responsive pressure sensitive adhesive in such formulation can vary between 10 to 99%, preferably between 30% to 75% and all ranges and values there between including ranges of 30% to 35%, 35% to 40%, 40% to 45%, 45% to 50%, 50% to 55%, 55% to 60%, 60% to 65%, 65% to 70%, and 70% to 75%. Such formulation can be prepared by physical blending of at least two different types of pressure sensitive adhesives. In one embodiment, a mixture of two of more photoresponsive adhesive can be blended together. Similarly, a mixture of two of more non-photoresponsive pressure sensitive adhesives can be used in photo-responsive pressure sensitive adhesive formulation. The photo-responsive pressure sensitive adhesive formulation can be prepared as a solvent borne, hot melt, or aqueous adhesives. In some more particular embodiments, the photo-responsive pressure sensitive adhesive formulation will contain additional components including but not limited to tackifier, plasticizer, surfactants, thickeners, defoamers. As an alternative to, or in addition to the non-photoresponsive pressure sensitive adhesive, the mixing and compounding can further include mixing the polymers of the photo-responsive pressure sensitive adhesive with an additive. The additive may include biocides, fillers, pigments, humectants, anti-oxidants, UV stabilizers or combinations thereof.

After the mixing and compounding, the produced photo-responsive pressure sensitive adhesive may be used for producing the wound dressings. In embodiments of the invention, the method of producing wound dressing 20 (shown in FIGS. 2A and 2B) may include coating the photo-responsive pressure sensitive adhesive to second surface 202 of a drape to form pressure sensitive adhesive layer 203 thereon. In some embodiments, a primer may be applied on second surface 202 before the coating step. In certain aspects, the coating may include substantially uniformly depositing the photo-responsive pressure sensitive adhesive to second surface 202.

In some more particular embodiments, the coating step may include directly coating the photo-responsive pressure sensitive adhesive onto the drape layer. Alternatively, or additionally, the coating step may include coating photo-responsive pressure sensitive adhesive onto releasable film 204, and transferring the coated photo-responsive pressure sensitive adhesive from the releasable film to second surface 202. In certain aspects, releasable film 204 may be coated with a silicone release coating thereon before coating the photo-responsive pressure sensitive adhesive thereon. In some more particular embodiments, the method of producing wound dressing 20 may further include applying a release coating on light filtering layer 205 such that light filter layer 205 can be releasably attached to first surface 201.

According to embodiments of the invention, the method of producing the wound dressings may further include laminating the drape (including first surface 201 and second surface 202), pressure sensitive adhesive layer 203, optionally releasable film 204, optionally light filtering layer 205 to form a laminated product. In some more particular embodiments, the absorbent layer may be laminated between the drape and the pressure sensitive adhesive layer. In embodiments of the invention, the method of producing wound dressing 20 may further include slitting the laminated product into desired shapes and/or sizes. The slit laminated product can further undergo packaging and sterilization to produce wound dressing 20.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods, and/or steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A photo-responsive pressure sensitive adhesive comprising:
a viscoelastic polymeric matrix of a polymer that includes at least a first polymeric segment and at least a second polymeric segment that are each covalently linked together through a photo-cleavable moiety.

2. The photo-responsive pressure sensitive adhesive of claim 1, wherein the photo-cleavable moiety is capable of photolytic cleavage of the covalent bonds formed between the moiety and the first and/or second polymer segments, respectively, in response to electromagnetic radiation having a wavelength of 300 nm to 500 nm.

3. The photo-responsive pressure sensitive adhesive of claim 2, wherein the wavelength is 300 nm to 400 nm, or preferably about 365 nm.

4. The photo-responsive pressure sensitive adhesive of any one of claim 1, wherein photo-cleavable moiety is a substituted aromatic nitro compound.

5. The photo-responsive pressure sensitive adhesive of claim 4, wherein the substituted aromatic nitro compound has the following structure: 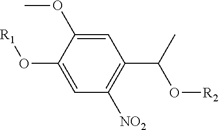
wherein R₁ and R₂ are each individually an acryloyl group, a methacryloyl group, an isocyanate group, or a hydroxyalkyl group that forms a covalent bond with the first polymeric segment and the second polymeric segment, respectively.

6. The photo-responsive pressure sensitive adhesive of claim 5, wherein the polymer has the following general structure: 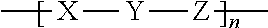
wherein X is the first polymeric segment, Y is the photo-cleavable moiety, Z is the second polymeric segment, and n is 1 to 10,000.

7. The photo-responsive adhesive of claim 5, wherein the photolytic cleavage breaks the R₁—O bond and/or the R₂—O bond.

8. The photo-responsive pressure sensitive adhesive of claim 1, wherein the first polymeric segment and the second polymeric segment each individually have a molecular weight of 10,000 g/mol to 1,000,000 g/mol.

9. The photo-responsive pressure sensitive adhesive of claim 1, wherein the first and second polymeric segments are each a viscoelastic polymer.

10. The photo-responsive pressure sensitive adhesive of claim 1, wherein the first polymeric segment is a viscoelastic polymer and the second polymeric segment is a non-viscoelastic polymer.

11. The photo-responsive pressure sensitive adhesive of claim 9, wherein the viscoelastic polymer is a polyacrylate, a polymethacrylate, a polyurethane, a hydrogel, a natural rubber, a synthetic rubber, a styrene block copolymer, a polyvinyl ether, a polyolefin, a hydrocolloid, a silicon or silicone based material, or a combination thereof.

12. The photo-responsive adhesive of claim 1, wherein the viscoelastic polymeric matrix is cytocompatible and/or biocompatible.

13. The photo-responsive adhesive of claim 1, wherein the viscoelastic polymeric matrix has a glass transition temperature (Tg) of −80° C. to 70° C., preferably −80° C. to 30° C. or 30° C. to 70° C.

14. The photo-responsive pressure sensitive adhesive of claim 1, wherein the photo-cleavable moiety is capable of photolytic cleavage of the covalent bonds formed between the moiety and the first and second polymeric segments, respectively, within 5 second to 1200 seconds in response to electromagnetic radiation.

15. A wound dressing comprising:
a first surface and an opposing second surface; and
a pressure sensitive adhesive layer comprising the photo-responsive pressure sensitive adhesive of claim 1 attached to at least a portion of the second surface of the wound dressing.

16. The wound dressing of claim 15, wherein the pressure sensitive adhesive layer comprises 10 g/m² to 200 g/m² of the photo-responsive pressure sensitive adhesive.

17. The wound dressing of claim 15, further comprising a releasable film attached to at least a portion of the pressure sensitive adhesive layer.

18. The wound dressing of claim 15, further comprising a light filtering layer comprising a pigment and/or a dye attached to at least a portion of the first surface, wherein the light filtering layer is configured to reflect and/or absorb electromagnetic radiation having a wavelength of 300 nm to 800 nm, preferably 400 nm to 800 nm, or more preferably 500 nm to 800 nm.

19. The wound dressing of claim 18, wherein the light filtering layer is transparent.

20. The wound dressing of claim 18, wherein the light filtering layer is opaque.

21. The wound dressing of claim 18, wherein the light filtering layer is releaseably attached to the first surface.

22. The wound dressing of claim 15, wherein the first surface is a drape layer.

23. The wound dressing of claim 15, further comprising an absorbent layer that is positioned between the second surface and the pressure sensitive adhesive layer.

24. A method of removing the wound dressing of claim 15 from a wound, the method comprising subjecting the wound dressing to electromagnetic radiation such that photolytic cleavage of one or more of the covalent bonds formed between the first polymeric segment and the photo-cleavable moiety and/or between the second polymeric segment and the photo-cleavable moiety occurs.

25. The method of claim 24, wherein the electromagnetic radiation is ultraviolet radiation.

26. The method of claim 25, wherein the electromagnetic radiation has a wavelength of 300 nm to 400 nm, or preferably about 365 nm.

27. The method of claim 24, wherein the tack of the pressure sensitive adhesive layer is reduced by at least 10% after being exposed to the electromagnetic radiation for 5 seconds to 1200 seconds.

28. The method of claim 24, wherein the electromagnetic radiation has an irradiance of 1 to 50 W/cm².
